(19) United States
(12) Patent Application Publication
(10) Pub. No.: US 2023/0364388 A1
(43) Pub. Date: Nov. 16, 2023

(54) CATHETER SECUREMENT DEVICE

(71) Applicant: ABIOMED, Inc., Danvers, MA (US)

(72) Inventor: Christopher Korkuch, Danvers, MA (US)

(73) Assignee: ABIOMED, Inc., Danvers, MA (US)

(21) Appl. No.: 18/196,022

(22) Filed: May 11, 2023

Related U.S. Application Data
(60) Provisional application No. 63/341,261, filed on May 12, 2022.

(51) Int. Cl. A61M 25/02

(52) U.S. Cl. CPC A61M 25/02

(57) ABSTRACT

An adapter for securing a catheter may include a housing composed of a first and second housing part and a locking mechanism, as well as a first flexible seal coupled to the first and second housing parts. The adapter has an “open” configuration where the first flexible seal is capable of being repositioned axially along a first catheter, cannula, or sheath having a first outer diameter. The adapter also has a “closed” configuration where the first flexible seal is incapable of being repositioned axially along the first catheter, cannula, or sheath without substantially deforming the first catheter, cannula, or sheath. The first flexible seal is configured to circumferentially surround an axial portion of only the first catheter, cannula, or sheath. The adapter can be coupled to a sheath hub to prevent at least axial motion of the adapter relative to the sheath hub.

### CROSS-REFERENCE TO RELATED APPLICATIONS

The present application claims priority to U.S. 63/341,261, filed May 12, 2022, the contents of which are incorporated by reference herein in its entirety.

### TECHNICAL FIELD

The present disclosure is related to the field of catheter securement, and in particular the prevention of movement of a medical device utilizing a catheter after a physician has properly positioned the device.

### BACKGROUND

Medical devices for treating patients often must be precisely placed within a patient to be effective, and to minimize the risk of complications. Such devices may be positioned at a distal end of a catheter, where the device has a larger diameter than the catheter. To position such devices in the patient, a physician may first insert an introducer sheath, and then insert the device through the introducer sheath until it is in a correct position. Depending on the needs of the treatment, the introducer sheath may need to be removed, and a repositioning sheath may need to be added. To keep the medical device in a correct position relative to the patient and maintaining sterility is extremely challenging. While fluoroscopic equipment can be used to correct placement in the event the device is moved, this can be time-consuming, which may be problematic for the treatment.

### BRIEF SUMMARY

An adapter for securing catheters is disclosed. The adapter may include a housing, comprising a first housing part coupled to a second housing part. The housing may also include a locking mechanism. The adapter may include a first flexible seal coupled to the first housing part and the second housing part. The housing may have a first (“open”) configuration where the first flexible seal is capable of being repositioned axially along a first catheter, cannula, or sheath having a first outer diameter, and a second (“closed”) configuration where the first flexible seal is incapable of being repositioned axially along the first catheter, cannula, or sheath without substantially deforming the first catheter, cannula, or sheath. The first flexible seal may be configured to circumferentially surround a portion of only the first catheter, cannula, or sheath.

In some embodiments, an end portion of the first housing part may be hingedly coupled to an end portion of the second housing part. In some embodiments, an inner surface of the first housing part may be slidably coupled to an outer surface of the second housing part. In some embodiments, the first flexible seal may be comprised of a thermoplastic elastomer or a synthetic rubber. In some embodiments, the first flexible seal may be comprised of comprised of a silicone or a polyisoprene.

In some embodiments, the adapter may include a second flexible seal coupled to the first housing part and the second housing part. The second flexible seal may be capable of being repositioned axially along a second catheter, cannula, or sheath when the housing is in the first configuration. The second flexible seal may be incapable of being repositioned axially along a second catheter, cannula, or sheath when the housing is in the second configuration without substantially deforming the second catheter, cannula, or sheath. The second catheter, cannula, or sheath may have a larger outer diameter than the first catheter, cannula, or sheath. The second flexible seal may be configured to circumferentially surround a portion of both the first catheter, cannula, or sheath and the second catheter, cannula, or sheath.

In some embodiments, the first catheter, cannula, or sheath has an outer diameter of 1-4 mm, and the second catheter, cannula, or sheath (if present) has an outer diameter of 4-8 mm.

In some embodiments, the adapter may include an attachment mechanism, configured to allow the housing to be coupled to a sheath hub, and prevent the adapter from moving relative to the sheath hub. In some embodiments, the attachment mechanism may be configured to be removably coupled to the sheath hub. In some embodiments, the attachment mechanism may include a protrusion or depression on a surface of the first housing part configured to interface with a depression or protrusion on an outer surface of the sheath hub.

In some embodiments, the locking mechanism comprises a depressible tab on one of the first housing part or second housing part configured to fit into a slot on the other of the first housing part or second housing part.

A system is also disclosed. The system may include an adapter as disclosed herein, an introducer sheath that includes a sheath hub coupled to the adapter, and an intravascular blood pump that includes a catheter in contact with the first flexible seal, where the catheter is incapable of sliding axially relative to the adapter when the adapter is in a closed configuration.

In some embodiments, the system may include an adapter as disclosed herein, an intravascular blood pump that includes a catheter in contact with the first flexible seal, where the catheter is incapable of sliding axially relative to the adapter when the adapter is in a closed configuration, and a repositioning sheath having a portion positioned around the catheter and in contact with a second flexible seal.

A kit is also disclosed. The kit may include an adapter as disclosed herein, an intravascular blood pump that includes a catheter; and an introducer sheath that includes a sheath hub. In some embodiments, the kit may also include a repositioning sheath.

A method for securing a catheter is also disclosed. The method may include positioning an adapter that includes a housing and a first flexible seal relative to a sheath hub of an introducer sheath and an intravascular blood pump that includes a first catheter such that the housing is capable of interacting with the sheath hub and the first flexible seal is capable of interacting with the first catheter. The housing may include a first housing part coupled to a second housing part, and may include a locking mechanism. The method may include reconfiguring the adapter from a first configuration to a second configuration when the adapter, sheath hub, and first catheter are correctly positioned. In the second configuration, the housing prevents the adapter from moving relative to the sheath hub and the first flexible seal circumferentially surrounds a portion of the first catheter and prevents the first catheter from moving relative to the adapter.

In some embodiments, prior to positioning the adapter, the method may include positioning a distal end of the introducer sheath in a patient, and inserting the intravascular blood pump through the introducer sheath and positioning the intravascular blood pump at a desired location in the patient. The first catheter may extend proximally beyond a proximal end of the sheath hub.

In some embodiments, the method may include positioning a distal end of the introducer sheath in a patient, attaching the adapter to the sheath hub, and prior to reconfiguring the adapter from a first configuration to a second configuration, inserting the intravascular blood pump through the introducer sheath and positioning the intravascular blood pump at a desired location in the patient. The first catheter may extend proximally beyond a proximal end of the sheath hub.

In some embodiments, the method may include reconfiguring the adapter from a second configuration to a first configuration and detaching the adapter from the sheath hub. In some embodiments, the method may include removing the introducer sheath from the patient. In some embodiments, the method may include inserting a repositioning sheath into the patient by sliding the repositioning sheath over the first catheter. In some embodiments, the method may include reconfiguring the adapter from a first configuration to a second configuration when the adapter, repositioning sheath, and first catheter are correctly positioned, the second configuration configured such that a second flexible seal circumferentially surrounds a portion of the first catheter and the repositioning sheath and prevents the adapter from moving relative to the repositioning sheath, and the first flexible seal circumferentially surrounds only a portion of the first catheter and prevents the first catheter from moving relative to the adapter.

### BRIEF DESCRIPTION OF DRAWINGS

FIG. 1 is a simplified embodiment of an adapter.

FIG. 2A is an illustration of an embodiment of an adapter in an open configuration.

FIG. 2B is an illustration of an embodiment of an adapter in a closed configuration.

FIG. 3A is an illustration of a side view of an embodiment of an adapter in an open configuration.

FIG. 3B is an illustration of a top view of an embodiment of an adapter in an open configuration.

FIG. 3C is an illustration of a side view of an embodiment of an adapter in a closed configuration.

FIG. 3D is an illustration of a top view of an embodiment of an adapter in a closed configuration.

FIG. 4 is an illustration of an adapter coupled to an introducer sheath with a sheath hub, an intravascular blood pump having a catheter, and a repositioning sheath.

FIG. 5 is a flowchart of an embodiment of a method.

### DETAILED DESCRIPTION

Disclosed is an adapter, system, kit, and method for securing catheters and medical devices, while maintaining sterility.

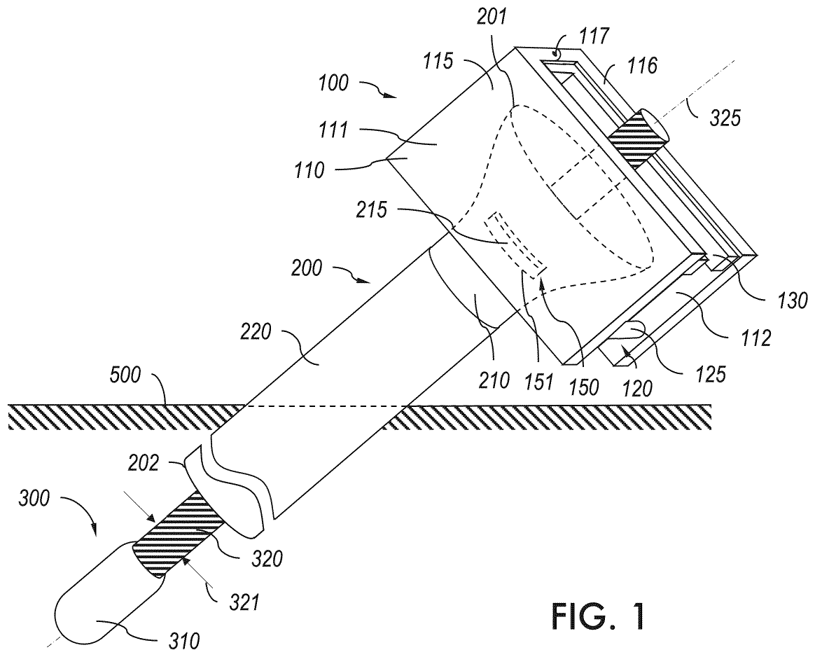

Referencing FIG. 1, an adapter 100 can be seen. The adapter may include a housing 110. In some embodiments, the housing may have an outer surface 111 and an inner surface 112, with a wall therebetween. The housing may have a first housing part 115 coupled to a second housing part 116. The housing may comprise a locking mechanism 120.

In some implementations, the housing may be formed of a rigid material such as 304 stainless steel. In some implementations, the housing may be formed of other rigid metals such as 316 stainless steel, or rigid polymers such as polyether ether ketone (“PEEK”), acrylonitrile butadiene styrene (“ABS”), or polycarbonate. In some implementations, the housing may be fully or partially coated, such as with a polymer.

In some embodiments, the adapter may have a first flexible seal 130 that is coupled to the first housing part 115 and the second housing part 116. In some embodiments, the flexible seal 130 is coupled to the inner surface 112 of the housing—that is, an inner surface of the first housing part and an inner surface of the second housing part.

The housing may have a first configuration where the first flexible seal 130 is capable of being repositioned axially 325 along a catheter, cannula, or sheath 320 having an outer diameter 321, and second configuration where the first flexible seal is incapable of being repositioned axially along the catheter, cannula, or sheath without substantially deforming or damaging the catheter, cannula, or sheath. In some embodiments, the catheter, cannula, or sheath 320 is coupled to a medical device 310, such as a blood pump.

In some embodiments, the flexible seal may be configured to circumferentially surround a portion of only a portion of only the catheter, cannula, or sheath 320. In some embodiments, the flexible seal may utilize a thermoplastic elastomer or a synthetic rubber. In some embodiments, the flexible seal may include a silicone or a polyisoprene.

In some embodiments, the adapter may include an attachment mechanism 150. The attachment mechanism may be configured to allow the housing 110 to be coupled to a sheath 200. In particular, the attachment mechanism may be configured to allow the housing 110 to be coupled to a sheath hub 210 and prevent the adapter 100 from moving relative to the sheath hub 210. A distal end of the sheath hub 210 may be attached to a proximal end of a sheath cannula 220, such that the sheath 200 is configured to slidably receive (through a lumen extending from a proximal end 201 to a distal end 202) the catheter, cannula, or sheath 320. In some embodiments, the adapter may only be prevented from moving axially 325 relative to the sheath hub. In some embodiments, the adapter may be prevented from moving axially and circumferentially relative to the sheath hub.

In some embodiments, the attachment mechanism 150 may be configured to be removably coupled to the sheath hub 210. In some embodiments, the attachment mechanism may be coupled to the sheath hub via, e.g., a pressure-sensitive adhesive.

In some embodiments, the attachment mechanism 150 may include a depression or extrusion 151 on a surface of the first housing part configured to interface with an extrusion or depression 215, as appropriate, on an outer surface of the sheath hub. In some embodiments, an extrusion on a surface of the sheath hub may be configured to fit through a slot or opening extending from an inner surface to an outer surface of the housing.

In some embodiments, the sheath cannula 220 of the sheath 200 may extend below a surface 500 of a patient's skin, and into a patient's vasculature (not shown).

The locking mechanism 120 for the adapter may be any appropriate locking mechanism. As shown in FIG. 1, the locking mechanism may include one or more pins 125 configured to extend from the inner surface 112 of the first housing part and/or the second housing part towards the other housing part, where it may interact with the other housing part.

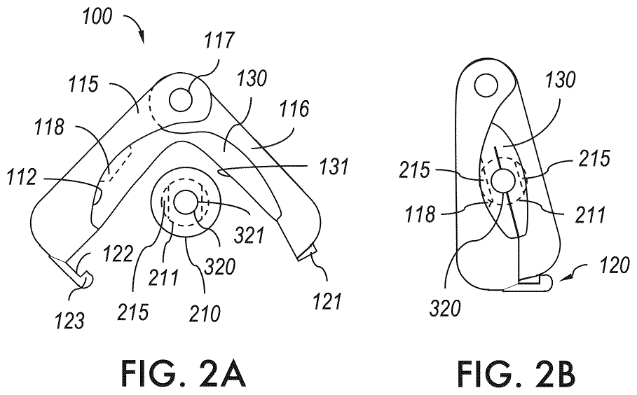

Referencing FIGS. 2A and 2B, in some embodiments, the locking mechanism 120 may include a tab 121 on one of the housing parts (here, the tab is shown on the second housing part 116), where the tab is configured to fit into a depression or slot 122 on the other housing part (here, the depression or slot is shown on the first housing part 115). In some embodiments, the depression or slot may be present on an extension portion 123 that extends from one housing part (here, the first housing part) towards the other housing part (here, the second housing part). In some embodiments, the locking mechanism may include a depressible tab (e.g., tab 121) on one of the first housing part or second housing part that is configured to fit into a slot 122 on the other of the first housing part or second housing part.

In FIG. 2A, one configuration (e.g., an “open” configuration) of the adapter 100 is shown. In this open configuration, the adapter 100 is configured to have two housing parts 115, 116 connected via a hinge 117. That is, in some embodiments, and end portion of the first housing part is hingedly coupled to an end portion of the second housing part.

In this open configuration, the adapter can be repositioned along or around the catheter, cannula, or sheath 320, and the sheath hub 210. As seen in FIG. 2A, there may be a depression 215 from an outer surface 211 of the sheath hub that is configured to interact with a protrusion 118 on the inner surface 112 of the housing. In some embodiments, one or more protrusions may be present on one of the two housing parts, such as the first housing part 115. In some embodiments, one or more protrusions may be on the inner surface of both housing parts.

Further, there may be an inner surface 131 of the flexible seal 130 that may be configured to interact with an outer surface 321 of the catheter, cannula, or sheath 320. In the open configuration, the flexible seal may not be in contact with the catheter, cannula, or sheath 320, or may only partially surround the catheter, cannula, or sheath.

In FIG. 2B, another configuration (e.g., a “closed” configuration) is shown. As seen, in this configuration, the locking mechanism 120 may be engaged, preventing the two housing parts from readily separating. As seen, in the closed configuration, the flexible seal 130 now substantially surrounds the catheter, cannula, or sheath 320. This creates a frictional force on the catheter, cannula, or sheath that holds it in place.

Further, protrusion 118 is interacting with the slot or depression 215 on the sheath hub. Given the interaction between the flexible seal and catheter, and the interaction between the protrusion 118 and the depression 215 in the sheath hub, the adapter cannot move at least axially, and the catheter cannot be adjusted at least in the axial direction with respect to the sheath hub. By unlocking the locking mechanism (and returning the adapter to the open configuration—see FIG. 2A—the catheter can be repositioned if necessary.

In some embodiments, the default configuration may be the closed configuration; that is, the user must manipulate the housing to change the adapter to its open configuration in order to, e.g., reposition the catheter, cannula, or sheath. When the housing is no longer manipulated, the adapter will return to the closed configuration. This may be done using, e.g., springs, elastomers, etc., as will be understood by those of skill in the art.

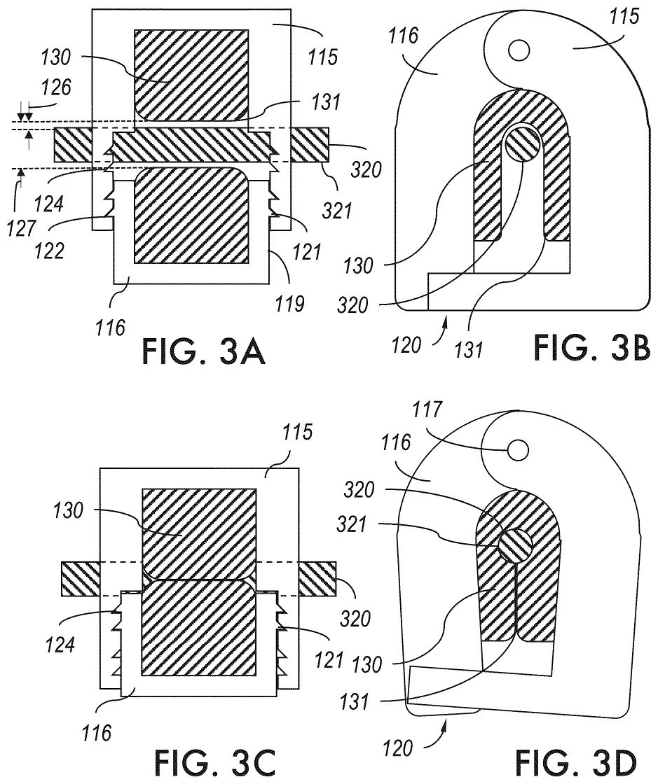

Referencing FIGS. 3A-3D, a different locking mechanism can be seen. In these figures, an inner surface of the first housing part may be slidably coupled to an outer surface of the second housing part. In some embodiments, the reverse may also be true—an inner surface of the second housing part may be slidably coupled to an outer surface of the first housing part.

In FIGS. 3A and 3B, an “open” configuration of a locking mechanism is seen. FIG. 3A shows a rotated side view, and FIG. 3B shows a top view of the arrangement. In some embodiments, the adapter may be configured such that in an open configuration, from above, the flexible seal 130 forms a “C” shape around the catheter, cannula, or sheath 320. The flexible seal 130 may be configured such that a separation distance 126 from an inner surface 131 of the flexible seal to an outer surface 321 of the catheter, cannula, or sheath from is at least 0.1, 0.2, 0.3, 0.4, or 0.5 mm up to 0.6, 0.7, 0.8, 0.9, or 1 mm, including all combinations and subranges thereof, with an equal separation distance on an opposing side of the catheter, cannula, or sheath.

In some embodiments, the flexible seal may be configured such that in the open configuration, a minimum separation distance 127 from an inner surface of a portion of the flexible seal coupled to a first housing part 115, in a direction normal to the inner surface, to an opposing inner surface of a portion of the flexible seal coupled to a second housing part 116 is provided. The separation distance 127 may be from 3.5 mm, 4 mm, or 4.5 mm up to 5 mm, 5.5 mm, or 6 mm, including all combinations and subranges thereof.

As seen in FIG. 3A, the locking mechanism may include one or more tabs or teeth 121 that are configured to interact with one or more depressions 122, 124. In some embodiments, the open configuration, the tabs or teeth 121 are configured to interact with a first set of depressions 122 of the housing part. In some embodiments, the tabs or teeth on one of the housing parts may be configured to easily allow movement towards the other housing part, but prevent movement away from the other housing part. In some embodiments, movement separating the first housing part from the second housing part may occur only if the sides 119 of one of the housing parts is squeezed or compressed.

In some embodiments, the locking mechanism may allow the housing to enter a closed configuration when the tabs or teeth are moved to engage a second set of depressions 124.

The closed configuration can be seen in FIGS. 3C and 3D, where a side view (FIG. 3C) and top view (FIG. 3D) are shown. As seen, in some embodiments, in the closed configuration, the flexible seal 130 circumferentially surrounds an axial portion of the catheter, cannula, or sheath 320. In some embodiments, the inner surface 131 of the flexible seal coupled to a first housing part 115 is in contact with at least a portion of the inner surface of the flexible seal coupled to the second housing part 116. As seen in FIG. 3D, in some embodiments, the portion of the flexible seal 130 where one inner surface is in contact with another may be located between the catheter, cannula, or sheath and the locking mechanism 120.

In some embodiments, in the closed configuration, the inner surface 131 of the flexible seal 130 may be in direct contact with substantially all (e.g., 95% or more) of the outer surface 321 of the catheter, cannula, or sheath 320 that the flexible seal circumferentially surrounds.

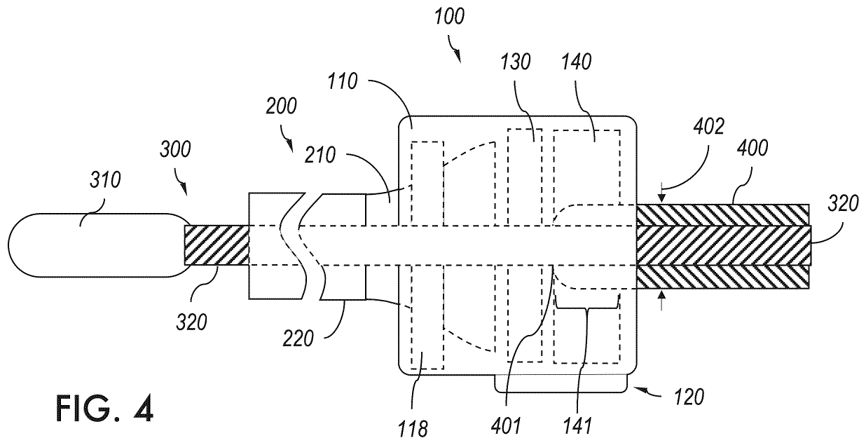

Referring to FIG. 4, in some embodiments, the adapter 100 may include a second flexible seal 140, configured in a manner similar to the first flexible seal 130.

However, as seen in FIG. 4, with two flexible seals, an adapter 100 can be used to prevent the axial motion of two catheters having different outer diameters relative to a sheath hub 210. Thus, in some embodiments, the second flexible seal 140 may be configured to hold a second catheter, cannula, or sheath 400 having a different outer diameter 402 than the diameter 321 of the catheter, cannula, or sheath 320 that the first flexible seal 130 is configured to hold.

The second flexible seal may be capable of being repositioned axially along a second catheter, cannula, or sheath when the housing is in the open configuration, and may be incapable of being repositioned axially along a second catheter, cannula, or sheath when the housing is in the closed configuration without substantially deforming the second catheter, cannula, or sheath.

In some embodiments, the second catheter, cannula, or sheath may have a larger outer diameter than the first catheter, cannula, or sheath. In some embodiments, the first catheter, cannula, or sheath 320 may have an outer diameter 321 of 1-4 mm, and the second catheter, cannula, or sheath 400 may have an outer diameter 402 of 4-8 mm.

In some embodiments, the second catheter, cannula, or sheath 400 extends at partially through the second flexible seal 140. In some embodiments, the second catheter, cannula, or sheath 400 extends at completely through the second flexible seal 140. In some embodiments, a distal end 401 of the second catheter, cannula, or sheath 400 may be positioned proximally from the first flexible seal 130, while the first catheter, cannula, or sheath 320 extends through the second flexible seal 140, the first flexible seal 130, the sheath hub 210, and the sheath cannula 220. In some embodiments, the adapter may be configured such that in the closed configuration, the second catheter, cannula, or sheath 400 have a portion 141 positioned around the first catheter, cannula, or sheath 320, the portion 141 being in contact with the second flexible seal 140.

In some embodiments, the sheath 200 may be an introducer sheath, the first catheter, cannula, or sheath 320 may be a catheter for a medical device, such as an intravascular blood pump comprising a catheter, and the second catheter, cannula, or sheath 400 may be a repositioning sheath.

Referring to FIG. 1, in some embodiments, a system may be provided. The system may include an embodiment of an adapter 100 as disclosed herein. The system may also include an introducer sheath 200 comprising a sheath hub 210. The sheath hub may be coupled to the adapter as disclosed herein. The system may also include an intravascular heart pump 300, comprising a catheter 320, the catheter in contact with the first flexible seal 130, incapable of sliding axially 325 relative to the adapter when the adapter is in the closed configuration.

Referring to FIG. 4, in some embodiments, a system may be provided that utilizes a repositioning sheath. Specifically, in some embodiments, the system may include an embodiment of an adapter 100 as disclosed herein. The system may also include an intravascular heart pump 300, comprising a catheter 320, the catheter in contact with the first flexible seal 130, incapable of sliding axially relative to the adapter when the adapter is in the closed configuration. The system may also include a repositioning sheath 400 having a portion 141 positioned around the catheter and in contact with the second flexible seal 140.

In some embodiments, the second flexible seal may be configured to circumferentially surround a portion 141 of both the first catheter, cannula, or sheath and the second catheter, cannula, or sheath.

In some embodiments, a kit is provided. Referring to FIG. 4, in some embodiments, the kit may include an embodiment of an adapter 100 as disclosed herein. The kit may also include an intravascular blood pump 300 comprising a catheter 320. In some embodiments, the blood pump 310 is located distal to the catheter 320. In some embodiments, the kit may include an introducer sheath 200 comprising a sheath hub 210. In some embodiments, the kit may include a repositioning sheath 400.

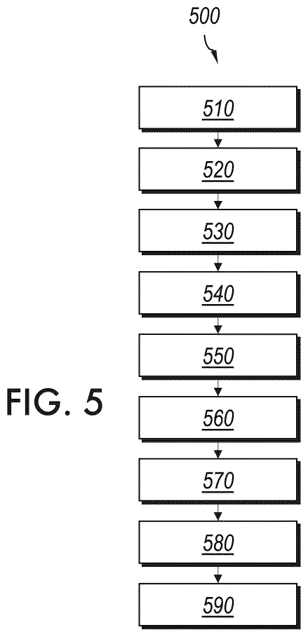

In some embodiments, a method for securing a catheter may be provided. As seen in FIG. 5, the method 500 may include positioning 510 a distal end of an introducer sheath, that has a sheath hub at a proximal end of the sheath, in a patient.

In some embodiments, the method 500 may also include inserting 520 an intravascular blood pump that comprises a first catheter through the introducer sheath and positioning the intravascular blood pump at a desired location in the patient. The first catheter may extend proximally beyond a proximal end of the sheath hub.

in some embodiments, the method 500 may include positioning 530 an embodiment of an adapter as disclosed herein relative to the sheath hub of the introducer sheath and the intravascular blood pump such that the adapter's housing may be able to interact with the sheath hub and the adapter's first flexible seal may be able to interact with the first catheter. The housing may include a first housing part coupled to a second housing part, and may include a locking mechanism.

In some embodiments, the method may include attaching 540 the adapter to the sheath hub. In some embodiments, this is done before the step of insertion 520. In some embodiments, this is done after the step of insertion 520.

The method 500 may also include reconfiguring 550 the adapter from a first configuration to a second configuration when the adapter, sheath hub, and first catheter are correctly positioned. As disclosed herein, the second configuration may be configured such that the housing prevents the adapter from moving at least axially relative to the sheath hub and the first flexible seal circumferentially surrounds an axial portion of the first catheter and prevents (e.g., via friction forces) the first catheter from moving relative to the adapter.

In some embodiments, the method 500 may include reconfiguring 560 the adapter from a closed configuration to an open configuration. In some embodiments, this may also include detaching the adapter from the sheath hub.

In some embodiments, the method 500 may include removing 570 the introducer sheath from the patient.

In some embodiments, the method 500 may include inserting 580 a repositioning sheath into the patient by sliding the repositioning sheath over the first catheter.

In some embodiments, the method 500 may include reconfiguring 590 the adapter from an open configuration to a closed configuration when the adapter, repositioning sheath, and first catheter, cannula, or sheath are correctly positioned. As disclosed herein, the closed configuration may be configured such that a second flexible seal may circumferentially surround a portion of both the first catheter, cannula, or sheath and the repositioning sheath, and prevents the adapter from moving relative to the repositioning sheath. The first flexible seal may circumferentially surrounds only a portion of the first catheter, cannula, or sheath, and prevents the first catheter from moving relative to the adapter.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Those skilled in the art will recognize or be able to ascertain using no more than routine experimentation many equivalents to the specific embodiments of the invention described herein. Such equivalents are intended to be encompassed by the following claims.

1. An adapter, comprising:
a housing, comprising a first housing part coupled to a second housing part, the housing comprising a locking mechanism;
a first flexible seal coupled to the first housing part and the second housing part;
wherein the housing has a first configuration where the first flexible seal is capable of being repositioned axially along a first catheter, cannula, or sheath having a first outer diameter, and second configuration where the first flexible seal is incapable of being repositioned axially along the first catheter, cannula, or sheath without substantially deforming the first catheter, cannula, or sheath; and
wherein the first flexible seal is configured to circumferentially surround a portion of only the first catheter, cannula, or sheath.

2. The adapter according to claim 1, wherein an end portion of the first housing part is hingedly coupled to an end portion of the second housing part.

3. The adapter according to claim 1, wherein an inner surface of the first housing part is slidably coupled to an outer surface of the second housing part.

4. The adapter according to claim 1, wherein the first flexible seal is comprised of a thermoplastic elastomer or a synthetic rubber.

5. The adapter according to claim 1, wherein the first flexible seal is comprised of silicone or polyisoprene.

6. The adapter according to claim 1, further comprising:
a second flexible seal coupled to the first housing part and the second housing part;
wherein the second flexible seal is capable of being repositioned axially along a second catheter, cannula, or sheath when the housing is in the first configuration,
wherein the second flexible seal is incapable of being repositioned axially along a second catheter, cannula, or sheath when the housing is in the second configuration without substantially deforming the second catheter, cannula, or sheath;
wherein the second catheter, cannula, or sheath has a larger outer diameter than the first catheter, cannula, or sheath; and
wherein the second flexible seal is configured to circumferentially surround a portion of both the first catheter, cannula, or sheath and the second catheter, cannula, or sheath.

7. The adapter according to claim 6, wherein the first catheter, cannula, or sheath has an outer diameter of 1-4 mm, and the second catheter, cannula, or sheath has an outer diameter of 4-8 mm.

8. The adapter according to claim 1, further comprising an attachment mechanism, configured to allow the housing to be coupled to a sheath hub, and prevent the adapter from moving relative to the sheath hub.

9. The adapter according to claim 8, wherein the attachment mechanism is configured to be removably coupled to the sheath hub.

10. The adapter according to claim 8, wherein the attachment mechanism comprises a protrusion or depression on a surface of the first housing part configured to interface with an depression or protrusion on an outer surface of the sheath hub.

11. The adapter according to claim 1, wherein the locking mechanism comprises a depressible tab on one of the first housing part or second housing part configured to fit into a slot on the other of the first housing part or second housing part.

12. A system, comprising:
an adapter according to claim 1;
an introducer sheath comprising a sheath hub, the sheath hub coupled to the adapter; and
an intravascular blood pump, comprising a catheter, the catheter in contact with the first flexible seal, incapable of sliding axially relative to the adapter when the adapter is in a closed configuration.

13. A system, comprising:
an adapter according to claim 1;
an intravascular blood pump comprising a catheter, the catheter in contact with the first flexible seal, incapable of sliding axially relative to the adapter when the adapter is in a closed configuration; and
a repositioning sheath having a portion positioned around the catheter and in contact with a second flexible seal.

14. A kit, comprising:
an adapter according to claim 1;
an intravascular blood pump comprising a catheter; and
an introducer sheath comprising a sheath hub.

15. The kit according to claim 14, further comprising a repositioning sheath.

16. A method for securing a catheter, comprising:
positioning an adapter comprising a housing and a first flexible seal relative to a sheath hub of an introducer sheath and an intravascular blood pump comprising a first catheter such that the housing is capable of interacting with the sheath hub and the first flexible seal is capable of interacting with the first catheter, the housing comprising a first housing part coupled to a second housing part, the housing comprising a locking mechanism; and
reconfiguring the adapter from a first configuration to a second configuration when the adapter, sheath hub, and first catheter are correctly positioned, where the second configuration configured such that the housing prevents the adapter from moving relative to the sheath hub and the first flexible seal circumferentially surrounds a portion of the first catheter and prevents the first catheter from moving relative to the adapter.

17-29. (canceled)
